(19) United States
(12) Patent Application Publication
(10) Pub. No.: US 2025/0170340 A1
(43) Pub. Date: May 29, 2025

(54) ELECTRONIC FLUID DISPENSER

(71) Applicant: Te Pari Products Limited, Oamaru (NZ)

(72) Inventor: PAUL FLEMING BUCKLEY, Auckland (NZ)

(21) Appl. No.: 18/955,109

(22) Filed: Nov. 21, 2024

(30) Foreign Application Priority Data
Nov. 29, 2023 (AU) 2023903845

(51) Int. Cl. A61M 5/315

(52) U.S. Cl. CPC A61M 5/31576; A61M 2005/31588; A61M 2250/00

(57) ABSTRACT

A variable dose fluid dispenser is described that may be used to deliver a dose of fluid to a subject. The fluid dispenser may comprise a dose adjustment method to control the volume of fluid dispensed. The fluid dispenser may comprise a plunger disposed within a chamber that translates in a linear direction relative to the chamber, translation moving the plunger: from a first position to a second position in a first direction causing fluid to be expelled from the chamber via the chamber outlet; and to a third position from the second position in a second opposite direction causing fluid to at least partly refill the chamber. Positional data from a sensor allows a controller to change the plunger positions to suit a dose to be administered.

### RELATED APPLICATIONS

This application derives priority from Australian patent application number 2023903845 filed on 29 Nov. 2023 with WIPO DAS code C5B9 incorporated herein by reference.

### TECHNICAL FIELD

An electronic fluid dispenser is described herein. The electronic fluid dispenser may dispense a variable dose of fluid and may be configured for delivering medicament to animals or other subjects.

### BACKGROUND

Farmers from time to time need to treat livestock with medicaments for controlling parasites and for the prevention and cure of diseases. These medicaments can be applied in a variety of ways including directly to the skin as a pour on liquid, orally as a drench, atomized as a nasal spray or as an intramuscular injection or subcutaneous injection.

Typically, the animals are held in a race or animal crush. A race or alley way is a narrow walkway with fencing on both sides that holds the animals in single file. The race leads from a holding pen and has gates at both ends to control the flow of animals. The farmer either walks along the side of the race and treats each animal or stands at the end of the race and treats each animal as it leaves the race. One problem with the race is that animals can jump or suddenly move their heads up, making injecting and oral drenching dangerous for both the animal and farmer. When animals are not restrained the risk of accidental injection injury during the injection process is high. Injection into a human of certain veterinary products can cause permanent injury and in severe cases death. An animal crush is an animal holding device, positioned at the end of the race and designed to hold individual animals in a more confined space. The crush has an entry gate and exit gate, where often the exit gate is a head bale designed to catch the animal and hold the animals head still. This helps restrain the animal although there is still risk of accidental injection injury.

The farming environment can be harsh. Extreme temperatures and high moisture levels are common. Many of the medicaments, in particular the pour on products, contain chemicals that can corrode electrical parts. Applicators are often dropped and damaged by livestock.

Most medicaments are applied using a manually operated applicator that automatically refills on the return stroke. Typically these applicators are hand held and have a spring loaded handle that is squeezed together to dispense medicament and refilled on the return stroke. Some of the medicaments are viscous or become viscous in cold weather and require considerable hand force to dispense. Other medicaments are applied in large dose sizes, also requiring considerable hand force to apply. One problem when treating large numbers of animals is operator hand fatigue. This can result in repetitive strain injury (RSI) for the operator. An additional problem is inaccurate dosing where the operator does not fully depress the applicator handle resulting in under size dosing of the animal. An additional problem when using viscous products is that the spring loaded handle can be slow to refill the applicator resulting in more time being required between animals. Powered applicators, both electrical, pneumatic or liquified petroleum gas (LPG) operated, have been developed to overcome the problems of hand fatigue however these can themselves be bulky and often require auxiliary cables or air supply hoses. Other powered applicators have expensive electronics and wireless connections to data bases that become too complex and unreliable in the farm environment.

It is useful to record the medicament and dose size alongside the animal electronic identity (EID) and date. This information can be useful in managing withholding periods for certain medicaments and in tracing animal health issues. One problem with manually operated applicators is there is no method to automatically record the medicament and dose size alongside the animal ID and date.

The amount of medicament applied to an animal can vary depending on the particular dose rate for each medicament. Some medicaments are of a fixed dose size where all the animals receive the same size dose. Other medicaments are applied relative to the weight of the animal. These medicaments require the farmer to change the dose size to suit each animal. The dose size can be displayed as a volume unit or as a weight unit or as combination of these units. One problem with some existing manually operated applicators and powered applicators is the dose size text is small and can be difficult to read.

Additional problems are the dose adjustment can be slow or difficult to change or the dose adjustment is easily knocked off setting, creating uncertainty for the farmer as to when the dose adjustment was knocked and which animals have been incorrectly dosed.

Another problem is that many of the applicators operate with a full chamber and the plunger only travels far enough in the chamber to dispense the required dose size. When injecting subjects, operators prefer to have the plunger travel the full length of the chamber and empty the chamber, for the operator to visually confirm that the entire dose size has been delivered to the subject. Applicators operate with a full chamber because it allows the dose size to be reduced without losing fluid. Many injectables are expensive and the full chamber method saves cost. Many injectables are hazardous and need to be disposed of in a controlled manner.

New Zealand patent no. 509851 describes a dispenser which attempts to overcome some of these problems. The hand piece has a keypad to alter the dose size and an alphanumeric display to show the dose size. The dose size is determined by counting the revolutions of the pump motor. This overcomes the problems of operator hand fatigue and offers limited recording capability however there is no safety provision, the electronics are complex and the dispenser is expensive.

U.S. Pat. No. 3,353,537 describes a pneumatically powered applicator where the dose size is set using a threaded adjustor and locking nut mounted on the rear of the applicator handle and the dose size is displayed on the side of the applicator. A disadvantage of this system is the dose adjustor is slow to use and requires a locking nut. Additionally the applicator has an air supply hose and is bulky.

WO2015/112027A2 describes a fluid dispenser that operates with a full chamber and has a magnet on the plunger that activates a fixed sensor on the PCB when the plunger is fully returned (home position). Dose volume is measured by counting the motor revolutions using an encoder mounted on the rear of the motor, with the plunger travelling forward the required distance for the selected dose volume. By operating with a full chamber the fluid dispenser is able to change dose volume without losing any fluid. However, the magnet and sensor of the fluid dispenser are not very accurate with the home position varying by up to +/−2 mm. Equally the encoder can miss counts and this positional drift needs to be reset using the home position.

Patent GB2503275 uses a linear potentiometer to measure the plunger position. The plunger has a sliding connection to the tracks of the potentiometer and voltage values are used to sense the position of the plunger. This method has a number of disadvantages. The connection and tracks wear due to the sliding of the wiper across the resistive element leading to a change in resistance, inaccuracy and failure. The method is difficult to seal against corrosion and is not suitable for a high repetition application. The dispenser uses a plunger with a valve on the plunger head and a barrel that forms a front chamber in front of the plunger head and a rear chamber behind the plunger head. When the plunger moves forward fluid is expelled from the front chamber and fluid is drawn in to the rear chamber. When the plunger returns, the fluid in the rear chamber moves thru the valve in the plunger head into the front chamber. This has the disadvantage of the motor needing to provide approximately double the power on the forward stroke to simultaneously empty the front chamber and draw fluid into the rear chamber. Also at the end of the dosing session, if the rear chamber is for example 5 ml but, the dose size only 1 ml, potentially 4 ml is wasted because air could be drawn into the front chamber.

It may be useful to provide a fluid dispenser configured to provide positional data from at least one sensor to allow a controller to change the start position and the stop position of the plunger, and be optimised to use as little fluid as possible and to reduce dose size without wasting or losing fluid, or at least provide the public with a choice.

Further aspects and advantages of the electronic fluid dispenser will become apparent from the ensuing description that is given by way of example only.

### SUMMARY

Described herein is a variable dose fluid dispenser that may be used to deliver a dose of fluid to a subject, such as a medicament to animal or person. The fluid dispenser may comprise a dose adjustment method to control the volume of fluid dispensed.

In a first aspect, there is provided a variable dose fluid dispenser used to deliver a dose of fluid to a subject, the variable dose fluid dispenser comprising:
a chamber with a longitudinal axis and comprising a chamber inlet and a chamber outlet, the chamber inlet and the chamber outlet fluidly communicating directly with the chamber;
   a plunger disposed within the chamber whereby the plunger translates in a linear direction relative to the chamber, translation in a same direction and common with a longitudinal axis of the chamber, translation moving the plunger:
      from a first position to a second position in a first direction causing fluid to be expelled from the chamber via the chamber outlet; and
      to a third position from the second position in a second opposite direction causing fluid to at least partly refill the chamber;
   a motor operatively coupled to the plunger, whereby the motor drives translation movement of the plunger;
   a trigger;
   at least one sensor;
   a controller in communication with the motor, the trigger and the at least one sensor; and
   whereby positional data from the at least one sensor allows the controller to change the first position and the third position of the plunger based on a desired dose volume;
   wherein:
the first position either equals the dose volume for the next dose, if the dose volume is equal or greater than the previous third position or, equals the previous third position if the dose volume is less than the previous third position;
the second position equals the first position less the dose volume; and
   the third position either equals the second position plus the dose volume if the second position is equal to zero or, equals the second position if the second position is greater than zero.

In a second aspect, there is provided a method of treatment of a subject comprising:
selecting a variable dose fluid dispenser substantially as described above;
providing a fluid dose in the chamber volume;
administering the fluid dose to the subject.

The above described variable dose fluid dispenser may provide a number of advantages over existing fluid dispensers. Examples of advantages found by the inventor may comprise one or more of the following:
High dose accuracy and consistent dosing;
No drift in dose settings hence accurate dosing;
The controller can measure the plunger position when the gun is turned on initially and does not need to perform a position re-set or initial calibration;
The home position can be set anywhere along the inductive field;
Since there is no mechanical contact to confirm plunger position, there is less or no maintenance or part wear and tear;
Due to the fluid dispenser design, the fluid dispenser is easy to seal.

### BRIEF DESCRIPTION OF THE DRAWINGS

Further aspects of the electronic fluid dispenser will become apparent from the following description that is given by way of example only and with reference to the accompanying drawings in which:

FIG. 1 shows a side elevation view of an example of the dispenser;

FIG. 2 shows a perspective view from above of the dispenser;

FIG. 3 shows a side cross-section elevation view of the dispenser;

FIG. 4 shows a cross-section plan elevation view of the dispenser;

FIG. 4a shows a front perspective view from above of the shroud lock rotated to the locked position;

FIG. 5 shows a diagram of controller in communication with an external device via a wireless link;

FIG. 6 shows a diagram of a controller in communication with the external device via the wireless link and an electronic identity reader (EID) integrated into the hand piece;

FIG. 7 shows a side elevation view of the dispenser configured to deliver a drench medicament;

FIG. 8 shows a flow diagram of one example of a method of use of the dispenser;

FIG. 9 shows the equations used to calculate the plunger position;

FIG. 10 is a table used to describe the plunger positions in the chamber and how the controller varies the plunger positions; and

FIG. 11 shows side views of a dispenser and a remote hand piece.

### DETAILED DESCRIPTION

As noted above, described herein is a variable dose fluid dispenser that may be used to deliver a dose of fluid to a subject, such as a medicament to animal or person. The fluid dispenser may comprise a dose adjustment method to control the volume of fluid dispensed.

For the purposes of this specification, the term ‘about’, or ‘approximately’, or ‘substantially’ and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1% to a reference quantity, level, degree, value, number, frequency, percentage, dimension, size, amount, weight or length.

The term ‘comprise’ and grammatical variations thereof shall have an inclusive meaning—i.e. that it will be taken to mean an inclusion of not only the listed components it directly references, but also other non-specified components or elements.

The term ‘fluid’ may be a gel to a liquid viscosity material and should not be limited to medicaments as the dispenser may be used to dispense other fluids.

### Fluid Dispenser

In a first aspect, there is provided a variable dose fluid dispenser used to deliver a dose of fluid to a subject, the variable dose fluid dispenser comprising:
a chamber with a longitudinal axis and comprising a chamber inlet and a chamber outlet, the chamber inlet and the chamber outlet fluidly communicating directly with the chamber;
   a plunger disposed within the chamber whereby the plunger translates in a linear direction relative to the chamber, translation in a same direction and common with a longitudinal axis of the chamber, translation moving the plunger:
      from a first position to a second position in a first direction causing fluid to be expelled from the chamber via the chamber outlet; and
      to a third position from the second position in a second opposite direction causing fluid to at least partly refill the chamber;
   a motor operatively coupled to the plunger, whereby the motor drives translation movement of the plunger;
   a trigger;
   at least one sensor;
   a controller in communication with the motor, the trigger and the at least one sensor; and
   whereby positional data from the at least one sensor allows the controller to change the first position and the third position of the plunger based on a desired dose volume;
   wherein:
the first position either equals the dose volume for the next dose, if the dose volume is equal or greater than the previous third position or, equals the previous third position if the dose volume is less than the previous third position;
the second position equals the first position less the dose volume; and
   the third position either equals the second position plus the dose volume if the second position is equal to zero or, equals the second position if the second position is greater than zero.

### Variable Volume in the Chamber

The above described fluid dispenser may operate with either a full chamber, or a chamber that is emptied on each stroke. That is, the chamber has a volume and when full of fluid to be dispensed, comprises one dose to be administered. The volume of fluid in the chamber volume may however be less than the chamber volume, this reduced volume of fluid corresponding to a smaller fluid dose. This smaller fluid dose may be proportional to the dose needed for example to a smaller patient or animal. The volume between doses may be varied therefore by altering the amount of fluid drawn into the chamber volume, potentially with each dose. The volume used may be determined by a controller that for example measures an animal characteristics e.g. animal ID, animal weight and so on, and then correlates the measured animal ID or weight to the dose needed and then signals to the plunger the plunger position required to move to a position that draws into the chamber volume, sufficient fluid to correctly dose the animal or subject relative to a fully empty chamber volume plunger position i.e. the plunger only moves sufficiently far back from a first position to a second position to draw in the volume of fluid commensurate with the dose.

Expressed another way, the controller may be configured to increase or reduce dose volume from a previous dose volume to a current dose volume without expelling fluid from the chamber outlet.

More specifically, dosing may occur as follows:
If a previous dose from the fluid dispenser was a fluid volume of 5 ml and the next dose a fluid volume of 3 ml, the plunger would travel forward stroke 3 ml to dispense the next dose and then, return stroke only 1 ml to be ready for a next 3 ml dose ensuring that, once the 3 ml dose is administered, the chamber volume is empty;
If a previous dose from the fluid dispenser was a fluid volume of 5 ml and the next dose a fluid volume of 1 ml, the plunger would travel forward stroke 1 ml to dispense the next dose and then, not complete any return stroke to be ready for a next 1 ml dose ensuring that no excess fluid is drawn in to the chamber volume;
If a previous dose from the fluid dispenser was a fluid volume of 5 ml and the next dose a fluid volume of 6 ml, the plunger would travel backwards 1 ml initially to draw a total volume of 6 ml into the chamber volume, then a forward stroke of 6 ml to dispense the next dose and empty the chamber volume and then complete a 6 ml return stroke to be ready for a next 6 ml dose ensuring that, once the 6 ml dose is administered, the chamber volume is empty.

As may be seen, the fluid dispenser is optimised to use as little fluid as possible and to always try to empty the fluid from the chamber volume.

The fluid dispenser is configured to reduce dose size without wasting or losing fluid.

### Fluid Entry Into the Chamber

Fluid is drawn into to the chamber volume independent to dose delivery. That is, fluid is only drawn into the chamber volume after delivery of a previous dose volume of fluid or prior to delivery of a dose of fluid from the fluid dispenser. Fluid is not drawn into the chamber volume during or simultaneous with delivery of the dose or expelling of fluid form the fluid dispenser.

### One Hand Piece

The chamber may be integrated into one hand piece or may be a sub-assembly releasably attached to the fluid dispenser hand piece.

The hand piece may be pistol shaped with the grip region offset from the fluid outlet by approximately 80 to 135 degrees.

The grip region of the hand piece may be offset from the fluid outlet by approximately 135 to 180 degrees.

The trigger lock and trigger may be separate parts. Alternatively the trigger lock and trigger may be combined into one part, whereby the first movement of the combined part releases the safety shroud and a continued movement of the combined part activates the trigger switch.

The shroud lock feature may be removed from the dispenser.

### Chamber

As noted above, the chamber include a chamber inlet and outlet that communicate directly with the chamber. The chamber may be a variable volume chamber.

### Plunger

As noted above, the plunger may be disposed within the chamber whereby the plunger translates in a linear direction relative to the chamber. Translation may be along a common longitudinal axis i.e. the chamber longitudinal axis is the same as the plunger translation direction longitudinal axis. Translation of the plunger may cause a change of volume in the chamber.

### Motor

As noted above, a motor may be used to drive plunger movement. The motor may be electrically powered. Power may be provided via a battery.

The motor may be approximately in-line with the longitudinal axis of a drive thread of the plunger. Alternatively the motor may be substantially parallel with a longitudinal axis of a drive thread of the plunger. In this example, the motor may rotate the drive thread via a gear or belt.

The motor may be at an angle to a longitudinal axis of the plunger. The motor may cause translation of the plunger via a bevelled gear operating on a drive rack. Alternatively, the motor may cause translation of the plunger via a belt or chain drive traveling substantially parallel to a longitudinal axis of the plunger. This method may allow the motor to be mounted in the grip portion of the fluid dispenser. A number of different ways may be used to achieve translation of the plunger.

The motor may be a 7.2V DC motor and the battery power source may be two lithium 18650 size batteries to provide approximately 7.2V. Alternatively, the motor could be any electrical motor capable of being mounted on the hand piece and the battery power source could be of any size or voltage output.

The battery power source may be mounted in the hand piece of the fluid dispenser.

Alternatively, power may be supplied by an external power source connected to the hand piece via a cable.

### Sensor

As noted above, at least one sensor may be used to determine a position of the plunger in the chamber.

A controller may be in communication with the at least one sensor and also the motor.

The plunger may linearly translate in the chamber from a first position to a second position to expel fluid from the chamber. The plunger may then linearly translate from a second position to a first position to draw fluid into the chamber. The extent of translation of the plunger between positions may be varied. Varying the distance of translation between the positions may vary the volume of fluid expelled and drawn into the chamber.

In some embodiments, the plunger may translate in the chamber from a first position to a second position to expel fluid from the chamber and from a second position to a third position to draw fluid into the chamber where varying the distance between the first position and the second position varies the volume of fluid expelled and varying the distance between the second position and the third position varies the volume of fluid drawn into the chamber.

The at least one sensor may be positioned adjacent the plunger.

The at least one sensor may be positioned alongside the plunger.

Positioning as noted above may assist the controller to determine the amount of fluid expelled and re-filled in the chamber volume.

The at least one sensor may span a length of travel of the plunger, whereby the at least one sensor provides positional data of the plunger to a controller.

The variable dose fluid dispenser may comprise multiple sensors, the multiple sensors configured as a row of multiple individual sensors.

Alternatively, or in combination with the above, the variable dose fluid dispenser may comprise a single long sensor, the single long sensor configured to provide multiple outputs.

The at least one sensor may be an inductive positional sensor. The sensor in this example may consist of a printed circuit board (PCB) with a sensor chip. The PCB and the sensor chip may be configured to trace or sense a metal target.

The sensor chip may send a signal to the controller of a position relative to the position of the metal target to the traces on the PCB.

There may be no sensor and/or encoder on the motor.

The controller may be in communication with the motor, the trigger and the at least one sensor, whereby the controller may determine a first position of the plunger, a second position of the plunger and a third position of the plunger, whereby the third position may be different to the first position and whereby the first position may be different to the third position. For example, the third position may be either:
the same as the first position; or
different to the first position

The at least one sensor may be a non-conductive positional sensor. The non-conductive positional sensor may be an inductive positional sensor.

The inductive positional sensor may be an inductive field. The inductive field may comprise tracks of copper on the printed circuit board (PCB).

The sensor and inductive field may be mounted or printed on a PCB.

The PCB may be positioned adjacent to the plunger. Fitting may be at a distance of 1-5, or 2-4, or approximately 3 mm from and/or adjacent to the plunger. The metal target may be positioned on the plunger. The metal target may be a metal member that creates induction. The metal may be ferrous or non-ferrous. The position of the metal target may be measured by the at least one sensor. The metal target on the plunger may be a small square of metal.

The fluid dispenser may operate with a single non-conductive sensor at the end of the expel stroke and a motor encoder used to count revolutions.

The fluid dispenser may operate with multiple non-conductive sensors. In this example, the multiple non-conductive sensors may be positioned at 0.05, or 0.1, or 0.2, or 0.5, or 1.0 ml increments, or similar. The multiple non-conductive sensors may be located adjacent to the plunger.

### Interface

A user may set a dose volume via an interface in communication with the controller.

The interface may allow a user of the fluid dispenser to adjust the distance between the first position and the second position and in turn, vary the dose expelled from the dispenser.

A visual means associated with the interface may display the amount of fluid to be expelled from the chamber. This may be calculated based on plunger position within the chamber. The visual means may be an electronic display or a graphic or series of graphics positioned on the plunger and/or chamber in such a manner as to indicate the amount of fluid to be expelled. The visual means may be displayed as a dose volume. Alternatively, the visual means may be displayed as a weight or, a combination of a volume and weight.

The dose volume may be adjusted via of a keypad. The keypad may have a power key, a mode key and up and down keys.

A trigger may activate a microswitch to activate dose delivery.

### Medicaments Dispensed

The fluid dispenser may be configured to dispense a variety of medicaments including but not limited to: a pour on liquid, an oral drench, a nasal spray and an injection.

### Injector Configuration

In an injector configuration, the dispenser may comprise a retractable safety shroud and needle optionally with a Luer taper. The safety shroud and needle may have a first safety shroud extended position and a second safety shroud retracted position.

In an extended position, the safety shroud may cover the needle. Covering may fully enclose at least the needle head and hence prevent the needle accidentally being caught on objects or a subject. In a retracted position, the safety shroud exposes the needle for injection of the subject.

The safety shroud may be spring loaded. In this example, the safety shroud may be biased to an extended position to cover the needle head. To administer a dose, the safety shroud, in the extended position, may be pushed against the subject against the bias to urge the safety shroud to retract and expose the needle head.

The safety shroud may have a shroud lock that locks the safety shroud in the extended position.

The safety shroud may activate a microswitch when travelling from the extended position to the retracted position. When the microswitch is activated, a signal is sent to the controller. The signal may activate the dose delivery from the dispenser via the needle. The signal may confirm to the controller that the safety shroud has been retracted. The controller may communicate that the safety shroud has been retracted and the dose has been delivered, deducing that the subject has been successfully injected.

### Fluid Dispensing Configuration

In a liquid dispensing configuration, the chamber outlet may comprise a nozzle. The nozzle may be configured to deliver the dose volume from the nozzle to the subject. The nozzle may comprise an elongated tube shape with an inlet that couples to the chamber outlet and a nozzle outlet from which fluid is ejected. The nozzle may be a spray nozzle.

As may be appreciated, this fluid dispensing configuration may be used for non-parenteral methods of administration such as for drench administration or trans-dermal administration.

### Interchangeable Dispensing

The fluid dispenser described may be interchangeable between the above configurations to allow for injection or fluid dispensing configurations to be used with the same fluid dispenser.

For example, the safety shroud, needle and Luer taper if present described above may be replaced with the nozzle described. Swapping configuration may be completed by removal and replacement of a cartridge or similar sub-assembly.

### Variable Input

The controller may receive a variable input communication and the controller may adjust the dose volume according to the variable input communication.

### EID Reader

The dispenser may further comprise an EID reader. The EID reader may be integrated into the hand piece.

The EID reader may send a signal based on the EID measured data to another device via a wireless communication link.

The EID reader and the controller may share the same wireless communication link. For example, the controller may measure the distance between the first position of the plunger and the second position of the plunger and, send a signal representing the volume of fluid dispensed to further device via a wireless communication link where the dispenser or further device may record the dose volume against the identity of a subject.

### Chamber

The chamber may be in fluid connection with an outlet. The outlet may be connected to the hand piece. The chamber may be removable from the outlet, leaving the outlet connected to the hand piece absent of the chamber.

### Manual Mode

In manual mode, if the fluid dispenser is set to empty the chamber on each stroke, when the dose volume is increased manually, the plunger moves back a commensurate amount to the dose volume, drawing more fluid into the chamber before the fluid dispenser dispenses the dose.

When the dose volume is decreased manually, and assuming an initial larger fluid volume dose is in the chamber volume, the plunger moves forward and expels unwanted fluid before the fluid dispenser dispenses the dose.

The fluid dispenser may have a keypad and, wherein pressing an up key or a down key may move the plunger a pre-set increment, dose volume would be printed on the side of the chamber to align with the head of the plunger. The dose may instead may be displayed on a display on the fluid dispenser and the dose adjusted via buttons, a touch screen or an app. As may be appreciated, the manual mode dose may be set and adjusted in a variety of ways.

### Automatic Mode

In automatic mode, the fluid dispenser or a controller thereof, may be linked to a weigh scale or other external input. Assuming the fluid dispenser is set to empty the chamber volume of fluid on each expel stroke, when the dose volume is decreased, the plunger may not move forward before the fluid dispenser is operated. Instead, when the fluid dispenser is operated, the plunger may only travel the required distance forward and may not empty the barrel, before returning to a new smaller dose home position.

An external device may set a dose volume via an external input (e.g. from a weight scale or EID reader) in communication with the controller.

There may be a number of variations possible to the above, such as the plunger always returning to the smallest dose, or the largest dose or a mid-point.

### EID Reader

The fluid dispenser may further comprise an EID reader. The EID reader may be integrated into the fluid dispenser. The EID reader may send a signal based on an EID measured data to an external device such as a processor via a wireless communication link.

### Remote Hand Piece

Optionally, a remote hand piece may be fluidly connected to the variable dose fluid dispenser. The remote hand piece may be controlled in dose size by the variable dose fluid dispenser.

For example, the remote hand piece may comprise a trigger, a safety shroud, a safety shroud microswitch, an EID reader and optionally, an LED display. When the remote hand piece trigger is actuated, dispensing may occur from the variable dose fluid dispenser and dispensed fluid is then transferred to the remote hand piece and dispensed from the remote hand piece.

Since driving of the plunger and dispensing of fluid may occur on the fluid dispenser, the remote hand piece may not comprise a motor or plunger.

### Method of Treatment

In a second aspect, there is provided a method of treatment of a subject comprising:
selecting a variable dose fluid dispenser substantially as described above;
providing a fluid dose in the chamber volume;
administering the fluid dose to the subject.

### Subject

The subject to which the fluid dose is administered may be a human or non-human animal. Non-human animals may include livestock or farmed mammals. Example of farmed mammals may include sheep, cattle, cows, deer, or pigs.

### Advantages

The above describe variable dose fluid dispenser may provide a number of advantages over existing fluid dispensers. Examples of advantages found by the inventor may comprise one or more of the following:
High dose accuracy and consistent dosing;
No drift in dose settings hence accurate dosing;
The controller can measure the plunger position when the gun is turned on initially and does not need to perform a position re-set or initial calibration;
The home position can be set anywhere along the inductive field;
Since there is no mechanical contact to confirm plunger position, there is less or no maintenance or part wear and tear;
Due to the fluid dispenser design, the fluid dispenser is easy to seal.

The embodiments described above may also be said broadly to consist in the parts, elements and features referred to or indicated in the specification of the application, individually or collectively, and any or all combinations of any two or more said parts, elements or features.

Further, where specific integers are mentioned herein which have known equivalents in the art to which the embodiments relate, such known equivalents are deemed to be incorporated herein as if individually set forth.

### EXAMPLES

The above described electronic fluid dispenser is now described by reference to specific examples with reference to the item numbering below:
100 Dispenser
1 Hand piece
1a Left hand part
1b Right hand part
2 Spring loaded shroud
2a Slot in mount for spring loaded shroud
3 Chamber
4 Outlet
5 Mount
6 Shroud lock
7 Trigger
8 Keypad
9 Battery holder
10 Motor
11 Gearbox
12 Drive thread
13 Plunger
14 Metal target
15 Sensor PCB
16 Needle
17 LEDs
18 Inlet
19 Outlet valve
20 Inlet valve
21 Controller
22 Trigger switch
23 Shroud switch
24 Rod
25 Battery pack
26 Lever
27 External device
28 Wireless link
30 Dose subject
31 EID Data
32 Electronic identity reader (EID)
35 Nozzle
36 Remote hand piece
37 Fluid hose
38 Electrical wire
39 Viewing chamber
40 Remote hand piece outlet
41 Remote hand piece trigger
42 Remote hand piece mount

### Example 1

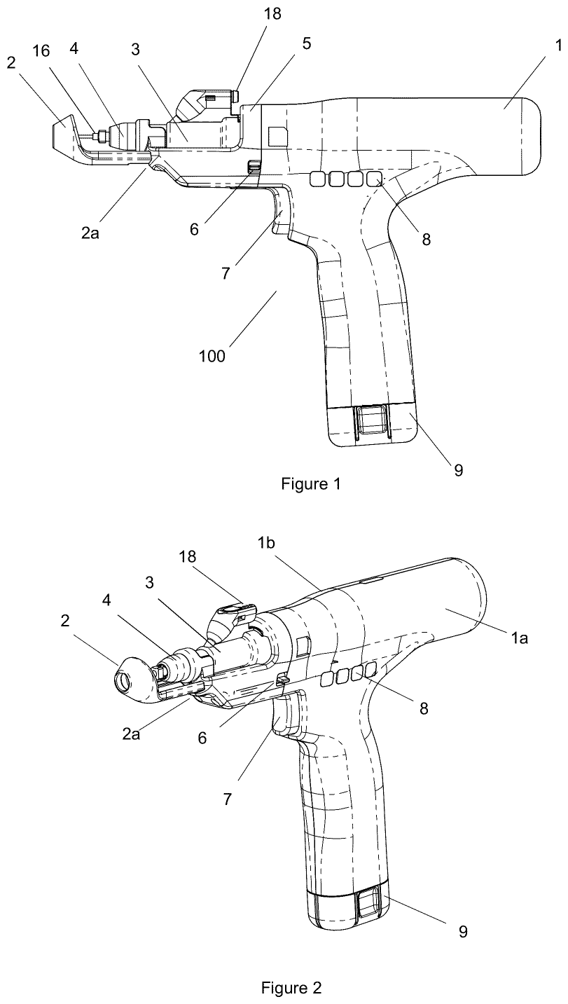

FIG. 1 and FIG. 2 show a side elevation view and perspective view from above of an example of the dispenser 100. The dispenser 100 as shown may comprise a gun shaped hand piece 1 consisting of a left hand part 1a and a right hand part 1b that are fixed together and used to locate and retain the mount 5.

A spring loaded shroud 2 is configured to slide in a slot 2a in the mount 5 under the chamber 3. The chamber 3 seals to the mount 5. An outlet 4 seals to the mount 5. Trigger 7 is configured to slide in the hand piece 1. Keypad 8 is shown with four keys consisting of a power key, up and down keys and a mode key. A battery holder 9 clips in to the hand piece 1.

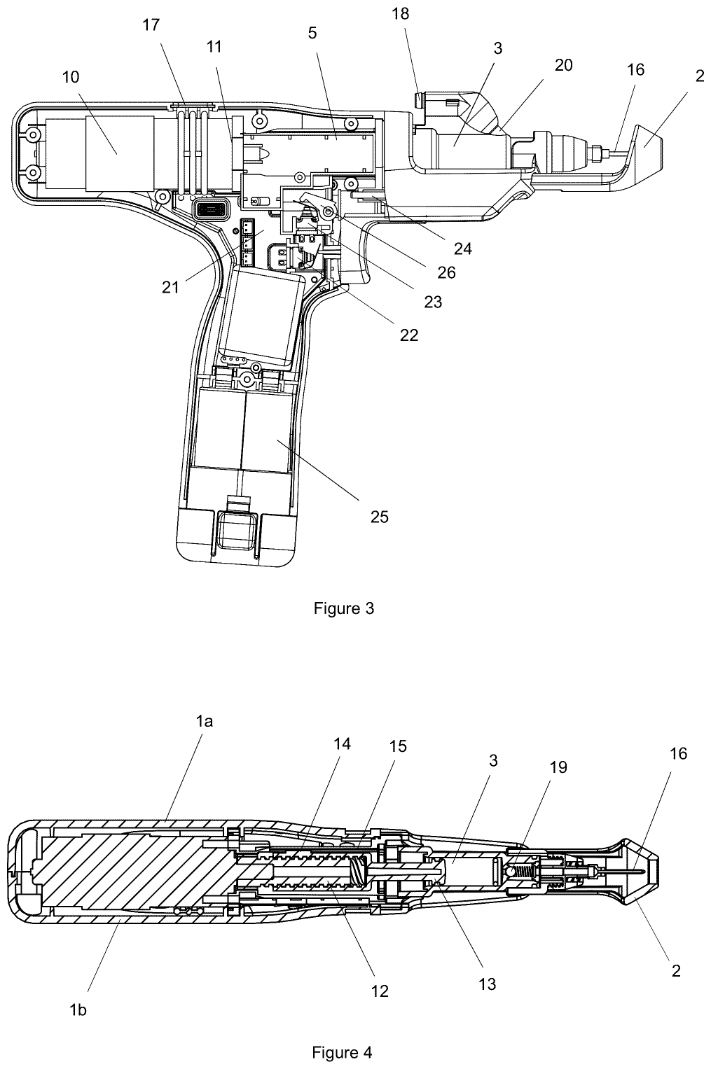

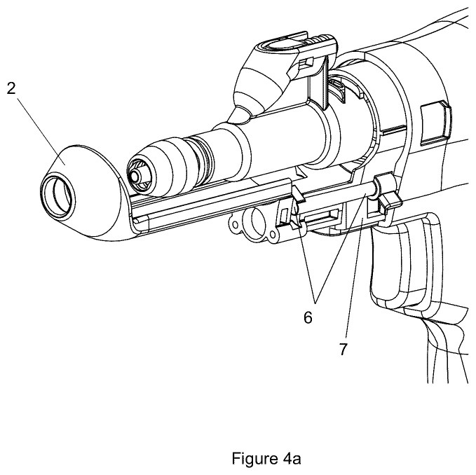

FIG. 3 shows a side cross-section elevation view. FIG. 4 shows a cross-section plan elevation view of the dispenser 100.

FIG. 4a shows a perspective view from in front and above the dispenser 100 with the shroud 2 lock 6 rotated to the locked position. In this position, a tab on the front of the shroud 2 lock 6 prevents the shroud 2 from being retracted and a tab at the rear of the shroud 2 lock 6 prevents the trigger 7 from moving and activating the trigger 7 switch 22.

As shown in FIG. 3 and FIG. 4, a motor 10 and gearbox 11 is engaged with a drive thread 12 that threads into a plunger 13 that is keyed to a non rotating part mount 5. The non rotating part mount 5 is configured to stop the plunger 13 rotating such that rotation of drive thread 12 results in the plunger 13 moving in a linear direction relative to the chamber 3 along a common longitudinal axis.

A metal target 14 is attached to the plunger 13 and a sensor PCB 15 is attached to the mount 5 adjacent to the plunger 13. The sensor PCB 15 measures the position of the target 14. The plunger 13 is shown in FIG. 3 and FIG. 4 in a first position.

As the plunger 13 moves from a first position to a second position, fluid (not shown) in the chamber 3 is expelled from the chamber 3 via the outlet 4 and needle 16.

As the plunger 13 moves from the second position to a third position fluid (not shown) is refilled into the chamber 3 via an inlet 18. An outlet valve 19 is positioned between the chamber 3 and the outlet 4. An inlet valve 20 is positioned between the inlet 18 and the chamber 3.

A controller 21 in communication with the sensor PCB 15 may control the motor 10. When the shroud 2 is in an extended position and the shroud lock 6 is in a locking position, a tab on the shroud lock 6 may prevent the shroud 2 from retracting and exposing the needle 16 and may prevent the trigger 7 from being operated.

The trigger 22 and a shroud switch 23 are connected to the controller 21. When the shroud 2 is retracted, rod 24 travels over lever 26 to activate shroud switch 23. Either the trigger 22 and/or the shroud switch 23 may send a signal to the controller 21 to start the motor 10. Battery pack 25 may provide power to the motor 10 and controller 21.

LEDs 17 may illuminate green, red and blue to act is indicia of particular actions.

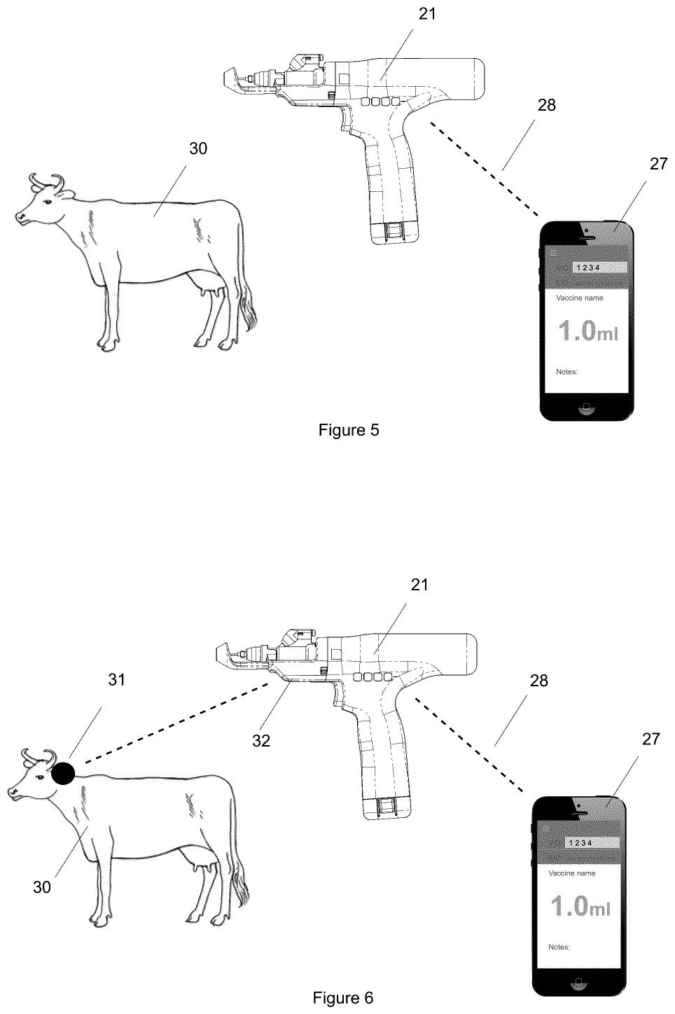

FIG. 5 shows a diagram of controller 21 in communication with an external device 27 via a wireless link 28 where the external device 27 sends input data to the controller 21 and the controller 21 controls the first, second and third positions of the plunger 13 to dose subject 30. The controller 21 may signal the external device 27 via the wireless link 28 that the dose has been delivered, where the external device 27 may record the dose volume against the identity of a subject 30. The external device 27 may record the medicament dispensed and the date dispensed against the subject 30. The controller 21 may record the dose volume, medicament and date.

FIG. 6 shows a diagram of a controller 21 in communication with the external device 27 via the wireless link 28 and an electronic identity reader (EID) 32 integrated into the hand piece 1. EID data 31 relating to the subject 30, is scanned and sent to an external device 27 via a wireless link 28. As shown in the flow diagram, the controller 21 may record the EID data 31. The EID 32 and the controller 21 may share the same wireless link 28. The external device 27 may also be common to both the EID 32 and the controller 21.

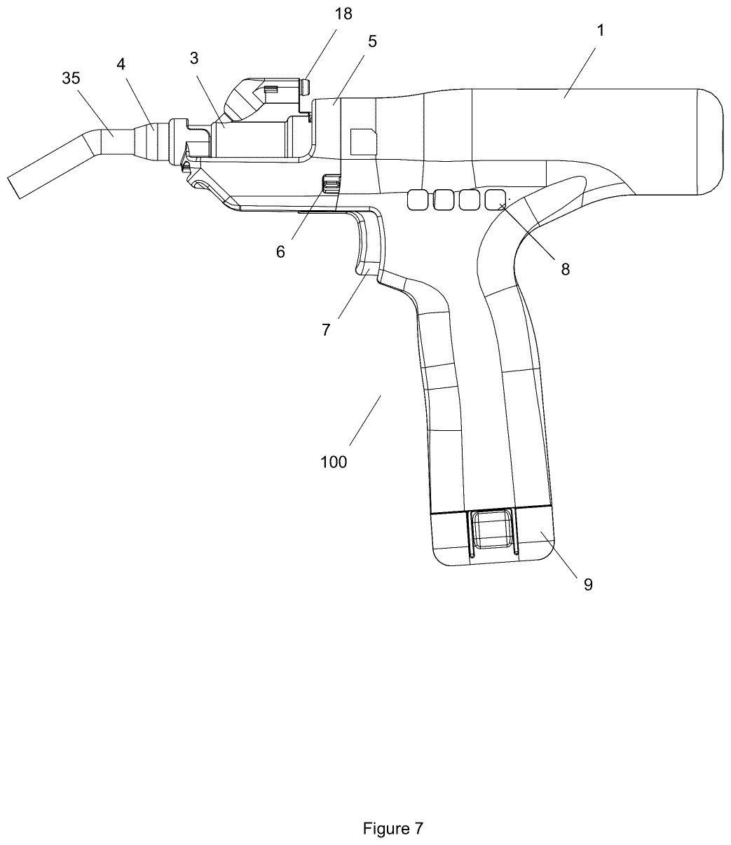

FIG. 7 shows a side elevation view of the dispenser 100 configured to deliver a drench medicament (not shown). The shroud 2 and needle 16 are replaced with a nozzle 35 that delivers fluid from the outlet 4 to the subject (not shown). In other respects, the dispenser 100 remains unchanged.

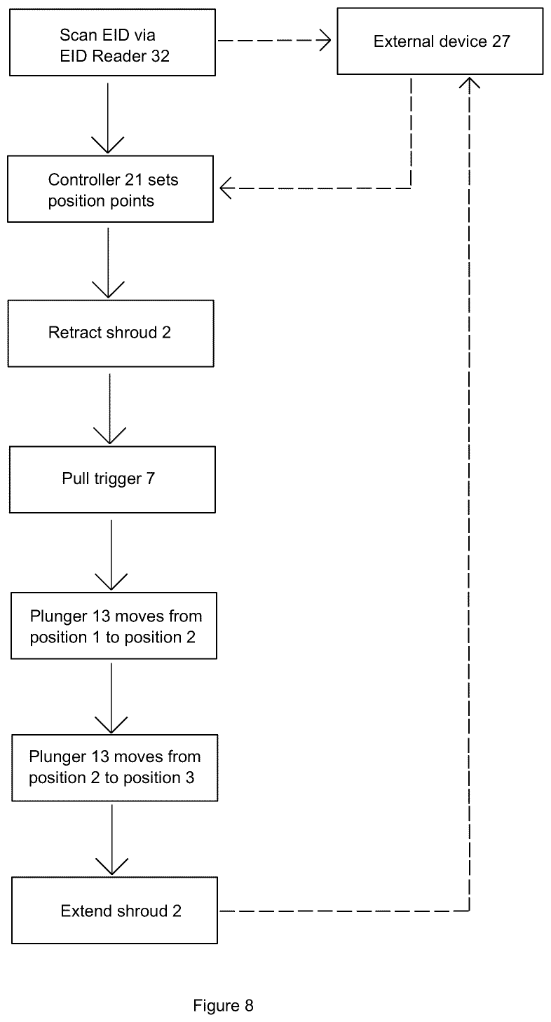

FIG. 8 shows a flow diagram of one example of a method of use of the dispenser 100 where the external device 27 sends input data relevant to the dose size. The transfer of data between the dispenser 100 and the external device 27 is shown as a dashed line and may be via a wireless link 28.

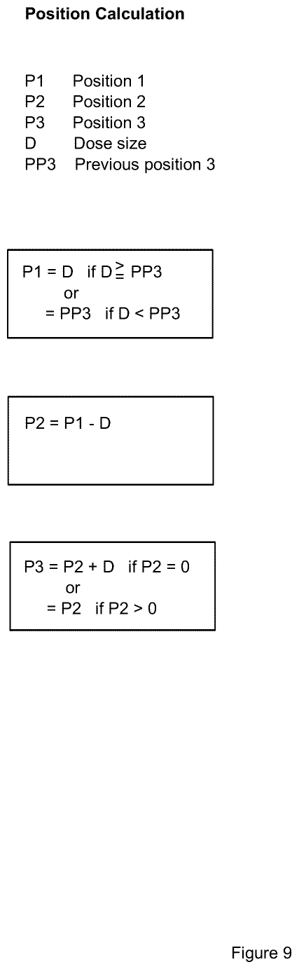

FIG. 9 shows the equations used to calculate the plunger 13 position. More specifically, FIG. 9 shows a calculation method that the controller may use to determine the position of the plunger in the chamber where:
Position 1 is the start position of the plunger.
Position 2 is the extended position of the plunger.
Position 3 is the stop position of the plunger.

Position 1 either equals the dose volume for the next dose, if the dose volume is equal or greater than the previous position 3 or, equals the previous position 3 if the dose volume is less than the previous position 3.

Position 2 equals position 1 less the dose volume.

Position 3 either equals position 2 plus the dose volume if position 2 is equal to zero or equals position 2 if position 2 is greater than zero.

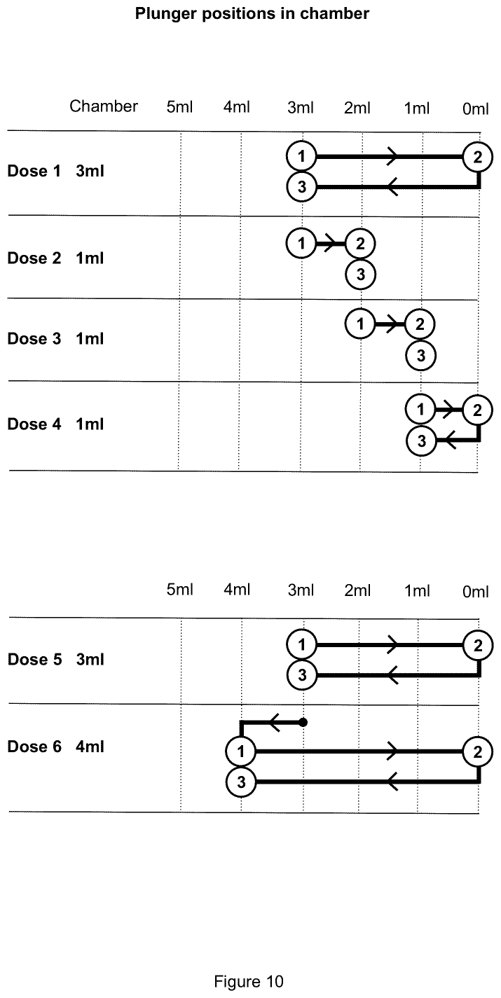

FIG. 10 is a table used to describe the plunger 13 positions in the chamber 3 and how the controller 21 varies the plunger 13 positions depending on whether one dose is larger or smaller than the preceding dose.
Example dose 1 as shown is 3 ml in volume, position 1 is at 3 ml, position 2 is 0 ml, position 3 is 3 ml.
Example dose 2 as shown is 1 ml in volume, position 1 is at 3 ml, position 2 is 2 ml, position 3 remains at 2 ml.
Example dose 3 as shown is 1 ml in volume, position 1 is at 2 ml, position 2 is 1 ml, position 3 remains at 1 ml.
Example dose 4 as shown is 1 ml in volume, position 1 is at 1 ml, position 2 is 0 ml, position 3 is 1 ml.

In the examples, dose 2 and dose 3, position 3 are different to position 1. In this method, the dispenser 100 is able to reduce the dose volume without wasting fluid and, by the third dose 1 ml, the plunger 13 is able to fully empty the chamber 3.
Example dose 5 as shown is 3 ml in volume, position 1 is at 3 ml, position 2 is 0 ml, position 3 is 3 ml.
Example dose 6 as shown is 4 ml in volume, position 1 is at 4 ml, position 2 is 0 ml, position 3 is 4 ml, where before the start of the dose, the controller 21 moves the plunger 13 from the previous position 3 at 3 ml to the new position 1 at 4 ml.

In example dose 6, position 1 is different to the previous position 3.

### Example 2

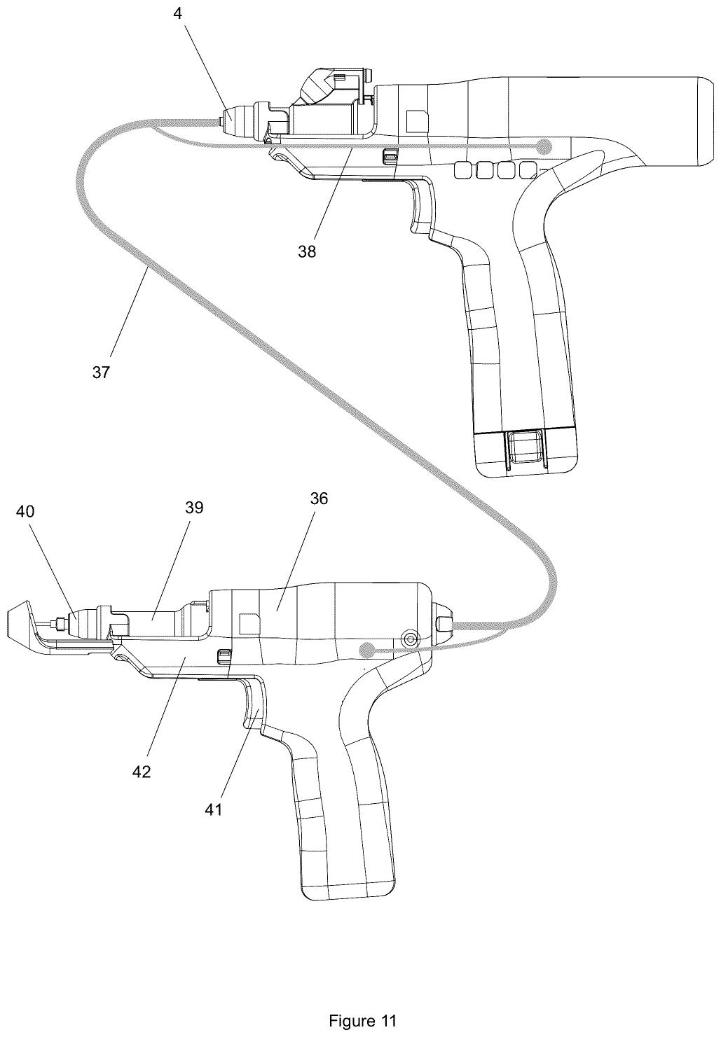

FIG. 11 shows side views of a dispenser 100 and a remote hand piece 36, the dispenser in this case acting like a remote pump to the remote hand piece 36. In this case, the remote hand piece 36 has a trigger 41 connected via a fluid hose 37 to the dispenser 100 and in communication via electrical wire 38 with the dispenser 100. Trigger 41 actuation on the remote hand piece 36 activates the dispenser 100 and fluid (not shown) is expelled via fluid hose 37 thru viewing chamber 39 and outlet 40 of the remote hand piece 36 to the subject (not shown).

The electrical wire 38 communication shown may be replaced with a wireless link communication (not shown).

The viewing chamber 39 may be in connection with the outlet 40. The outlet 40 may be connected to the remote hand piece 36 and remote hand piece mount 42. The viewing chamber 39 is removable from the outlet 40, leaving the outlet 40 connected to the remote hand piece 36.

Aspects of the electronic fluid dispenser have been described by way of example only and it should be appreciated that modifications and additions may be made thereto without departing from the scope of the claims herein.

What is claimed is:

1. A variable dose fluid dispenser used to deliver a dose of fluid to a subject, the variable dose fluid dispenser comprising:
a chamber with a longitudinal axis and comprising a chamber inlet and a chamber outlet, the chamber inlet and the chamber outlet fluidly communicating directly with the chamber;
a plunger disposed within the chamber whereby the plunger translates in a linear direction relative to the chamber, translation in a same direction and common with a longitudinal axis of the chamber, translation moving the plunger:
from a first position to a second position in a first direction causing fluid to be expelled from the chamber via the chamber outlet; and
to a third position from the second position in a second opposite direction causing fluid to at least partly refill the chamber;
a motor operatively coupled to the plunger, whereby the motor drives translation movement of the plunger;
a trigger;
at least one sensor;
a controller in communication with the motor, the trigger and the at least one sensor; and
whereby positional data from the at least one sensor allows the controller to change the first position and the third position of the plunger based on a desired dose volume;
wherein:
the first position either equals the dose volume for the next dose, if the dose volume is equal or greater than the previous third position or, equals the previous third position if the dose volume is less than the previous third position;
the second position equals the first position less the dose volume; and
the third position either equals the second position plus the dose volume if the second position is equal to zero or, equals the second position if the second position is greater than zero.

2. The variable dose fluid dispenser of claim 1 wherein, the controller is configured to increase or reduce dose volume from a previous dose volume to a current dose volume without expelling fluid from the chamber outlet.

3. The variable dose fluid dispenser as claimed in claim 1 wherein, the at least one sensor is located adjacent to the plunger and the at least one sensor spans a length of travel of the plunger, whereby the at least one sensor provides positional data of the plunger to a controller.

4. The variable dose fluid dispenser as claimed in claim 1 wherein, the variable dose fluid dispenser comprises multiple sensors, the multiple sensors configured as a row of multiple individual sensors.

5. The variable dose fluid dispenser as claimed in claim 1 wherein, the variable dose fluid dispenser comprises a single long sensor, the single long sensor configured to provide multiple outputs.

6. The variable dose fluid dispenser as claimed in claim 1 wherein, the at least one sensor is an inductive positional sensor, the at least one sensor comprising a printed circuit board (PCB) with a sensor chip, the PCB and the sensor chip configured to trace or sense a metal target.

7. The variable dose fluid dispenser as claimed in claim 6 wherein, the metal target is a non-ferrous metal member that creates induction and the position of which is measured by the at least one sensor.

8. The variable dose fluid dispenser as claimed in claim 1 wherein, the controller is in communication with the motor, the trigger and the at least one sensor, whereby the controller determines a first position of the plunger, a second position of the plunger and a third position of the plunger, whereby the third position is either:
the same as the first position; or
different to the first position.

9. The variable dose fluid dispenser as claimed in claim 1 wherein, a user sets a dose volume via an interface in communication with the controller.

10. The variable dose fluid dispenser as claimed in claim 1 wherein, an external device sets a dose volume via an external input in communication with the controller.

11. The variable dose fluid dispenser as claimed in claim 1 wherein, fluid is only drawn into the chamber volume after delivery of a previous dose volume of fluid or prior to delivery of a dose volume of fluid from the fluid dispenser and fluid is not drawn into the chamber volume during or simultaneous with delivery of the dose volume or expelling of fluid from the fluid dispenser.

12. The variable dose fluid dispenser as claimed in claim 1 wherein, the fluid dispenser comprises an injector configuration, the fluid dispenser having a retractable safety shroud and needle.

13. The variable dose fluid dispenser as claimed in claim 1 wherein, the fluid dispenser comprises a liquid dispensing configuration wherein the chamber outlet comprise a nozzle, the nozzle configured to deliver the dose volume from the nozzle to the subject.

14. The variable dose fluid dispenser as claimed in claim 1 wherein, the fluid dispenser comprises an EID reader, the EID reader integrated into the variable dose fluid dispenser and wherein the EID reader sends a signal based on an EID measured data to an external device via a wireless communication link.

15. The variable dose fluid dispenser as claimed in claim 1 wherein, a remote hand piece is fluidly connected to the variable dose fluid dispenser, the remote hand piece comprising a trigger, a safety shroud, a safety shroud microswitch, an EID reader and optionally, an LED display and, when the remote hand piece trigger is actuated, dispensing occurs from the variable dose fluid dispenser and dispensed fluid is then transferred to the remote hand piece and dispensed from the remote hand piece.

16. A method of treatment of a subject comprising:
selecting a variable dose fluid dispenser, the variable dose fluid dispenser comprising:
a chamber with a longitudinal axis and comprising a chamber inlet and a chamber outlet, the chamber inlet and the chamber outlet fluidly communicating directly with the chamber;
a plunger disposed within the chamber whereby the plunger translates in a linear direction relative to the chamber, translation in a same direction and common with a longitudinal axis of the chamber, translation moving the plunger:
from a first position to a second position in a first direction causing fluid to be expelled from the chamber via the chamber outlet; and
to a third position from the second position in a second opposite direction causing fluid to at least partly refill the chamber;
a motor operatively coupled to the plunger, whereby the motor drives translation movement of the plunger;
a trigger;
at least one sensor;
a controller in communication with the motor, the trigger and the at least one sensor; and
whereby positional data from the at least one sensor allows the controller to change the first position and the third position of the plunger based on a desired dose volume;
wherein:
the first position either equals the dose volume for the next dose, if the dose volume is equal or greater than the previous third position or, equals the previous third position if the dose volume is less than the previous third position;
the second position equals the first position less the dose volume; and
the third position either equals the second position plus the dose volume if the second position is equal to zero or, equals the second position if the second position is greater than zero; and
providing a fluid dose in the chamber volume at the first position;
administering the fluid dose to the subject to the second position; and
moving the plunger to the third position.

17. The method as claimed in claim 16 wherein the subject to which the fluid dose is administered is a non-human animal.

18. The method as claimed in claim 16 wherein the subject to which the fluid dose is administered is selected from: sheep, cattle, cows, deer, or pigs.
